(19) United States
(12) Patent Application Publication
(10) Pub. No.: US 2026/0355251 A1
(43) Pub. Date: Nov. 12, 2026

(54) COMPOSITION FOR PROMOTING HAIR GROWTH

(71) Applicants: Idea Therapeutics SL, Alcobendas (ES); Instituto Médico Estético De Madrid, S.L., Madrid (ES); Fundación para la Investigación Biomédica del Hospital Clínico San Carlos (IDISSC), Madrid (ES)

(72) Inventors: Eduardo López Bran, Madrid (ES); Pilar Tornero Esteban, Madrid (ES); Lorena Pozo Pérez, Madrid (ES); José Ángel Sánchez García, Alcobendas (ES); Marisa Berenguer Muñoz, Alcobendas (ES)

(73) Assignees: Idea Therapeutics SL, Alcobendas (ES); Instituto Médico Estético De Madrid, S.L., Madrid (ES); Fundación para la Investigación Biomédica del Hospital Clínico San Carlos (IDISSC), Madrid (ES)

(21) Appl. No.: 19/714,949

(22) Filed: Jun. 22, 2026

Related U.S. Application Data
(63) Continuation of application No. PCT/EP2024/088205, filed on Dec. 20, 2024.

(30) Foreign Application Priority Data
Dec. 22, 2023 (EP) 23383357.3

(51) Int. Cl. A61K 35/28; A61K 8/60; A61K 8/98; A61K 31/7076; A61P 17/14; A61Q 7/00

(52) U.S. Cl. CPC A61K 35/28; A61K 8/606; A61K 8/981; A61K 31/7076; A61P 17/14; A61Q 7/00

(57) ABSTRACT

It refers to a composition for promoting hair growth, which comprises isolated and expanded MSCs, preferably adipose derived stem cells, ATP and a pharmaceutically acceptable vehicle. It further refers to a kit of parts comprising individual vials with MDCs and ATP. It refers also to said composition or said kit for use in androgenic alopecia. It further refers to cosmetic or non-therapeutic use of said composition or said kit for promoting hair growth. It further refers to isolated and expanded MSCs, preferably adipose derived stem cells, for use in promoting hair growth and for the prevention and/or treatment of androgenic alopecia.

### RELATED APPLICATIONS

This application is a continuation of international application number PCT/EP2024/088205, filed Dec. 20, 2024, the entire contents of which are incorporated herein by reference.

### FIELD OF THE INVENTION

The present invention relates to the field of compositions for promoting hair growth, in particular for promoting hair growth in androgenic alopecia.

### BACKGROUND ART

Androgenetic alopecia (AGA) is a progressive hair loss disorder that affects both sexes all over the world. AGA patients suffer localized hair loss and it is graded in men by the Norwood scale according to the hair loss horseshoe pattern. In women such hair loss occurs in a dispersed manner over the mid-frontal scalp towards the anterior frontal line and it is measured by the Savin scale, as disclosed in Dinh et al., Clin. Interv. Aging, 2007, 2 (2), 189-199.

AGA aetiology is caused by dihydroxytestosterone (DHT), an androgen derived from testosterone when it is accumulated in the scalp. DHT has a higher affinity towards androgen receptors in the hair follicles and AR gene is overexpressed, as disclosed in Ellis et al., Exp. Rev. Mol. Med., 2002, 4 (22), 1-11, and in Urysiak-Czubatka et al., Postep. Derm. Alergol., 2014, 31 (4), 207-215. Hair become miniaturised and ultimately falls out, as disclosed in Urysiak-Czubatka et al. op. cit., and in Otberg et al. Endocrinol. Metab. Clin. N. Am., 2007 36 (2), 379-398.

Male AGA is clearly an androgen-dependent condition, and a genetic predisposition is observed, whereas in female AGA, the role of androgens is still uncertain, as disclosed in Kelly et al., Drugs, 2016, 76, 1349-1364.

AGA affects quality of life and self-esteem of patients and their expectations about therapy results is usually higher than reality; for this reason, it is important to clarify that the main treatment goal is to stop progression and prevent further hair thinning, as disclosed in Kelly et al., op. cit.

AGA is a dynamic and progressive disease, therefore after initial diagnosis it is important to quickly apply not only a treatment that inhibits the progression of the disease, but also one that can reverse the changes that have already occurred, as disclosed in the review of Krefft-Trzciniecka et al., Cells, 2023, 12, 951 (doi, org/10.3390).

There are several therapies available for the treatment of AGA. Treatment with 5-alpha reductase inhibitors (e.g., finasteride, dutasteride), minoxidil, latanoprost and ketoconazole are already disclosed, even the combination of finasteride and minoxidil is most commonly used. Other current treatment options include laser therapy, scalp microneedling, hair mesotherapy, hair transplantation, platelet-rich plasma injections (see Kelly et al., op. cit.).

In P. T. Rose, Clin. Cosm. Invest. Dermatol., 2015, 8, 361-370, it is disclosed that hair transplant surgery became an excellent means to treat many forms of male and female hair loss, although there was room for the results to be enhanced.

In Epstein et al., Facial Plats. Surg. Clin. N. Am., 2018, 26 (4), 503-511, it is disclosed the treatment of men and women suffering hair loss by administering either adipose injections alone or with a 4:1 mixture of adipose tissue to platelet-rich plasms (PRP).

According to Krefft-Trzciniecka et al., op. cit., the use of stem cells in AGA seems to be a promising alternative to the standard treatment or it could play a role of complementary therapy to improve the effect of primary treatment. Adipose tissue is a well-known source of precursors and stem cells, and a variety of regenerative products can be generated from adipose tissue, including nano fat, stromal vascular fraction (SVF), mesenchymal stem cells (MSCs), adipose-derived stem cell conditioned medium (ADSC-CM), and extracellular vesicles (EV).

ADSC-CM is by far the most used stem cells-based therapy.

In Park et al., Biomed. Res., 2010, 31 (1), 26-34, it is disclosed that the secretomes of adipose-derived stem cells (ADSCs) mediate diverse skin-regeneration effect, and the authors concluded that they promote hair growth.

In Fukuoka et al., Am. J. Cosm. Surg., 2012, 29 (4), 273-282, it is disclosed that ADSCs secrete cytokines and growth factors, such as keratinocyte growth factor, vascular endothelial growth factor, platelet-derived growth factor and hepatocyte growth factor, which are very important for hair growth.

In Shin et al., Int. J. Dermatol., 2015, 54 (6), 730-735, it is disclosed that the application of ADSC-CM showed efficacy in treating female pattern hair loss, wherein the ADSC-CM was collected after culturing the ADSCs under hypoxic conditions and total protein production quantitated.

In Fukuoka et al., eplasty, 2015, 65-72, it is disclosed the use of a commercially available product containing a protein solution from ADSC-CM, and authors observed that said treatment increased hair numbers both in male and female patients. Said results were confirmed later by the authors in Fukuoka et al., Curr. Stem Cell Res. Med., 2017, 12 (7), 531-534.

In the review of Shin et al. Curr. Stem Cell Res. Med., 2017, 12 (7), 524-530, it is disclosed that treatment with ADSC-CM comprising ADSC-related proteins can stimulate hair growth.

In Won et al., Curr. Stem Cell Res. Med., 2017, 12 (7), 535-543, it is disclosed that ADSC-CM comprising ADSCs derived proteins induces the anagen phase and promotes hair growth in mice and enhances the elongation of hair shafts in ex vivo human hair organ cultures.

In Perez-Meza et al., Stem Cells Cloning, 2017, 10, 1-10, it is disclosed the use of a composition comprising purified autologous fat graft (Puregraft) and adipose-derived regenerative cells (Kerastem Celution), for the treatment of patients with hair loss. Said combination was administered by injection in the subcutaneous tissue of the scalp.

In Gentile et al., Stem Cell Investig., 2017, 4 (doi: 10.21037/sci.2017.06.04), it is disclosed the use of the system Rigenera® to obtain stem cells from patients' scalp biopsies without culturing (human follicle stem cells, HFSCs), to be administered using subcutaneous injections.

In Elmaadawi et al., J. Dermatol. Treat., 2018, 29 (5), 431-440, it is disclosed the use of autologous bone marrow derived mononuclear cells (including stem cells) for the treatment of resistant cases of AA and AGA.

In DE-A-4323616 it is disclosed the use of nucleic acids, their building blocks or their derivatives for regenerating hair, for reducing hair loss and for stimulating hair growth.

In KR-A-2016-0050898 it is disclosed a synergistic action between ATP and adenosine on the hair growth of hair follicle cultures.

Thus, in spite of different alternatives in the art for promoting hair growth in AGA, most of them using ADSC-CM and SVF, there is a need for efficient compositions.

### SUMMARY OF INVENTION

An aspect of the present invention relates to a composition for promoting hair growth.

Another aspect of the invention is a kit of parts.

Another aspect of the invention is said composition or said kit for use for use in the prevention or treatment of alopecia.

Another aspect of the invention is the cosmetic or non-therapeutic use of said composition for promoting hair growth.

Another aspect of the invention is isolated and expanded mesenchymal stem cells, for use in promoting hair growth and for the prevention and/or treatment of androgenic alopecia.

### FIGURES

FIG. 1 shows mice after being depilated.

FIG. 2 shows the results at day 21 of the administration of the pharmaceutically acceptable vehicle (HypoThermosol® FRS Preservation Media, HTS) alone, which is the control group (Group 1) of Example 5.

FIG. 3 shows the results at day 21 of the administration of ADSCs with ATP comprising a mixture of non-encapsulated ATP and liposomated ATP (Group 5) of Example 5.

FIG. 4 shows the results at day 21 of the administration of ADSCs with non-encapsulated ATP (Group 6) of Example 5.

### DESCRIPTION OF THE INVENTION

The present invention relates to a composition for promoting hair growth comprising Mesenchymal Stem Cells (MSCs), preferably isolated and expanded MSCs, more preferably isolated and expanded Adipose Stem Cells (ADSCs), and ATP (adenosine triphosphate), wherein ATP is preferably comprised in the composition, expressed in concentration of ATP, at a concentration of at least 10 μM, and wherein the composition further comprises a pharmaceutically acceptable vehicle, preferably an aqueous pharmaceutically acceptable vehicle wherein the MSCs are dispersed and the ATP is dissolved.

More particularly, the present invention relates to a therapeutically effective amount of isolated and expanded mesenchymal stem cells, preferably adipose derived stem cells (ADSC), a therapeutically effective amount of ATP, and a pharmaceutically acceptable vehicle, wherein the therapeutically effective dose of ATP is comprised in the composition, expressed in concentration of ATP, at a concentration between about 10 μM and about 20 mM.

As used herein, Mesenchymal stem cells (MSCs) are a class of multipotent stromal cells derived from the mesodermal lineage, characterized by their ability to self-renew and to differentiate into a variety of specialized cell types-most notably osteoblasts (bone), chondrocytes (cartilage), myocytes (muscle), and adipocytes (fat). These cells are widely distributed throughout the body and can be isolated from several tissues, including bone marrow, umbilical cord, dental pulp, and adipose tissue. Adipose-derived stem cells (ADSCs) are a population of MSCs specifically isolated from adipose (fat) tissue. While sharing the core properties of MSCs, including surface marker expression and multipotent differentiation potential, ADSCs are particularly abundant and readily accessible, making them a valuable source of MSCs for research and clinical applications. In essence, ADSCs are a specialized subset of MSCs, illustrating the broader principle that MSCs can be obtained from multiple tissue reservoirs.

As used herein, the term “about” typically indicates a permissible variation of +10% from the stated numerical value. In other words, if a particular parameter is described as “about X,” it generally encompasses values ranging from 0.9× to 1.1×. This allowance acknowledges normal measurement and manufacturing tolerances, as well as small fluctuations that may occur in practicing the invention.

The inventors of the present invention have thus developed a new composition for promoting hair growth, which surprisingly shows an improved, even synergistic, effect between the component's MSCs, preferably ADSCs, and ATP.

In the present description, as well as in the claims, the singular forms “a”, “an” and “the” include the plural reference unless the context clearly indicates otherwise. The term “about” refers to a deviation of plus/minus 10%, preferably plus/minus 5%. The percentages are expressed in % by weight (wt %), unless stated the contrary. The ranges defined by the terms “between . . . and . . . ” or by the terms “from . . . to . . . ” are meant to include also said stated endpoints thereof, and they also include any narrower sub-range.

The expression “a therapeutically effective amount” means the amount of a compound that, when administered to a patient for treating a disease, is sufficient to provide a therapeutic effect. An amount that is effective in therapy is an amount which produces a biological activity and will depend, among other things, on the individual. Therefore, the “therapeutically effective amount” will vary depending on the compound, the disease and its severity and the age, weight, etc., of the patient to be treated.

The expression “pharmaceutically acceptable” means that it is useful in preparing a pharmaceutical composition and is generally safe, non-toxic, and neither biologically nor otherwise undesirable and includes that is acceptable for veterinary use as well as human pharmaceutical use.

In the context of the invention, the term “dose” refers to a specified amount of medication taken at one specific time, and the term “dosage” involves a specific amount, number, and frequency of doses over a specific period of time.

The term “confluence (%)” is conventionally used in the art to express a cell density (degree of saturation) per area, and a unit usually used in experiments by those of ordinary skill in the art to relatively express the number of cells (cell density) per unit area in cell culture.

### Hair Growth Composition

One aspect of the present invention is a composition for promoting hair growth, preferably an aqueous composition, comprising, preferably a therapeutically effective dose of, mesenchymal stem cells (MSCs) and ATP (adenosine triphosphate), wherein ATP is comprised in the composition at a concentration, expressed in concentration of ATP, of at least 10 μM, and wherein the composition further comprises a pharmaceutically acceptable vehicle, preferably an aqueous pharmaceutically acceptable vehicle wherein the MSCs are dispersed and the ATP is dissolved.

In another embodiment of the first aspect of the invention, preferably in combination with any of the previous or subsequent embodiments, the ATP is comprised in the composition, expressed in concentration of ATP, at a concentration between 10 μM and 20 mM, preferably at a concentration between 50 μM and about 200 μM or about 250 M, preferably at a concentration between 75 μM and about 200 μM or about 250 μM, still more preferably at a concentration of about 100 μM, still more preferably at a concentration of 100 μM. Nevertheless, the concentration and type of ATP can be present in the composition in any of the types or further values or ranges indicated in the section entitled “ATP”.

In another embodiment of the first aspect of the invention, preferably in combination with any of the previous or subsequent embodiments, the MSCs are derived from adipose tissue, bone marrow, umbilical cord, placenta, amniotic fluid, and peripheral blood, preferably from adipose tissue.

In another embodiment of the first aspect of the invention, preferably in combination with any of the previous or subsequent embodiments, the MSCs are present in the composition in a concentration of between 4×10⁴ and 15×10⁷ cells per mL of the pharmaceutically acceptable vehicle, preferably in a concentration between 10⁵ and about 10⁶ cells per mL of the pharmaceutically acceptable vehicle, preferably in a concentration between 1×10⁶ cells per mL and 20×10⁶ cells per mL of the pharmaceutically acceptable vehicle. Nevertheless, the cells can be present in the composition in any of the further values or ranges indicated in the section entitled “Mesenchymal stem cells”.

In another embodiment of the first aspect of the invention, preferably in combination with any of the previous or subsequent embodiments, the MSCs are isolated and expanded mesenchymal stem cells, preferably isolated and expanded mesenchymal stem cells derived from adipose tissue, bone marrow, umbilical cord, placenta, amniotic fluid, or peripheral blood.

In another embodiment of the first aspect of the invention, preferably in combination with any of the previous or subsequent embodiments, the mesenchymal stem cells are adipose derived stem cells (ADSCs), preferably isolated and expanded adipose derived stem cells (ADSC).

In another embodiment of the first aspect of the invention, the composition comprises a therapeutically effective amount of isolated and expanded adipose derived stem cells, a therapeutically effective amount of ATP, and a pharmaceutically acceptable vehicle; wherein preferably ATP is comprised in the composition at a concentration, expressed in concentration of ATP, of at least 10 μM. Preferably, the ATP is comprised in the composition, expressed in concentration of ATP, at a concentration between 10 μM and 20 mM, preferably at a concentration between 50 μM and 250 M, preferably at a concentration between 75 μM and about 250 μM, still more preferably at a concentration of about 100 μM, and still more preferably at a concentration of 100 μM. Also preferably, MSCs are present in the composition in a concentration of between 4×10⁴ and 15×10⁷ cells per mL of the pharmaceutically acceptable vehicle, preferably in a concentration between 10⁵ and about 10⁶ cells per mL of the pharmaceutically acceptable vehicle, preferably in a concentration between 1×10⁶ cells per mL and 20×10⁶ cells per mL of the pharmaceutically acceptable vehicle. It is, however, noted that the cells can be present in the composition in any of the further values or ranges indicated in the section entitled “Mesenchymal stem cells” in combination with any of the values or ranges indicated in the section entitled “ATP”.

As indicated in the examples of the present invention, the combination of MSCs, more preferably ADSCs, and ATP into a hair growth composition provides an improved, even synergistic effect, wherein the hair growing effect of the combination is higher than the effect of the components when applied separately.

Generally, the composition for promoting hair growth comprises a liquid pharmaceutically acceptable vehicle, preferably an aqueous liquid pharmaceutically acceptable vehicle, wherein the MSCs, preferably isolated and expanded MSCs, more preferably isolated and expanded ADSCs, are dispersed, and ATP is dissolved.

In an embodiment, the composition of the invention is an aqueous composition, which comprises water as pharmaceutically acceptable vehicle, usually the content of water is not less than 90% (v/v), preferably not less than 95% (v/v).

In further embodiments of the hair growth composition of the invention, the MSCs, preferably ADSCs, are present in the composition in a therapeutically effective amount between 1×10⁶ cells per mL and 20×10⁶ cells per mL of the pharmaceutically acceptable vehicle, preferably in a therapeutically effective amount between 10⁵ and about 10⁶ cells per mL of the pharmaceutically acceptable vehicle, and preferably the ATP is present in the composition in a therapeutically effective amount, expressed in concentration of ATP, between about 20 μM and about 10 mM, preferably in a concentration between 50 μM and 250 μM, preferably at a concentration between 75 μM and about 250 μM, still more preferably at a concentration of 100 μM.

In further embodiments of the hair growth composition of the invention, the composition comprises about 10⁶ MSCs, preferably ADSCs per mL of the pharmaceutically acceptable vehicle, and the ATP is present in the pharmaceutically acceptable vehicle at concentration between about 75 μM and about 200 μM or about 250 μM, more preferably the ATP is present in the pharmaceutically acceptable vehicle at concentration of about 100 μM.

In further embodiments of the hair growth composition of the invention, the MSCs are isolated and expanded ADSCs, wherein the ADSCs are present in a therapeutically effective amount between 1×10⁶ cells per mL and 20×10⁶ cells per mL of the pharmaceutically acceptable vehicle, preferably in a therapeutically effective amount between 10⁵ and about 10⁶ cells per mL of the pharmaceutically acceptable vehicle and preferably the ATP is present in the composition in a therapeutically effective amount, expressed in concentration of ATP, between about 20 μM and about 10 mM, preferably in a concentration between 50 μM and about 200 μM or about 250 μM, preferably at a concentration between 75 μM and about 200 μM or about 250 μM, still more preferably at a concentration of 100 μM.

In further embodiments of the hair growth composition of the invention, the therapeutically effective dose of isolated and expanded ADSCs is comprised between about 4×10⁴ ADSCs and about 15×10⁷ ADSCs per mL of the pharmaceutically acceptable vehicle, preferably in a therapeutically effective amount between 10⁵ and about 10⁶ cells per mL of the pharmaceutically acceptable vehicle, and the therapeutically effective dose of ATP is comprised, expressed in concentration of ATP, between about 10 μM and about 20 mM.

In further embodiments of the hair growth composition of the invention, the composition comprises about 10⁶ isolated and expanded ADSCs per mL of the pharmaceutically acceptable vehicle, and a concentration of ATP comprised between about 75 μM and about 200 μM or about 250 μM, preferably 100 μM.

It is noted that further useful combinations of cells and ATP in the present invention can be found by combining the values or ranges indicated in the section entitled “Mesenchymal stem cells” with the values or ranges indicated in the section entitled “ATP”.

### Mesenchymal Stem Cells

The composition of the invention comprises a therapeutically effective amount of mesenchymal stem cells, preferably mesenchymal stem cells derived from adipose tissue, bone marrow, umbilical cord, placenta, amniotic fluid, or peripheral blood; more preferably the composition of the invention comprises a therapeutically effective amount of isolated and expanded mesenchymal stem cells, more preferably isolated and expanded ADSCs

As already indicated, Mesenchymal stem cells (MSCs), also known as mesenchymal stromal cells or medicinal signaling cells, are multipotent stromal cells that can differentiate into a variety of cell types, including adipocytes (fat cells), osteoblasts (bone cells), chondrocytes (cartilage cells), and myocytes (muscle cells).

MSCs are found throughout the body: adipose tissue, bone marrow, cord cells, molar cells, and amniotic fluid.

Although bone marrow was the original source of MSCs, adipose derived stem cells (ADSCs) can be obtained in larger quantities and are easier and safer to isolate than bone-marrow derived MSCs. As disclosed in Strioga et al., Same or not the same? Comparison of adipose tissue-derived versus bone marrow-derived mesenchymal stem and stromal cells, Stem Cells Dev., 2012, 2724-52, ADSCs seem to be as effective as bone marrow MSCs in clinical application.

Therefore, in a preferred embodiment, the MSCs are ADSCs. ADSCs may be isolated and expanded according to the procedure disclosed in prior art, for example in Park et al., Hair growth stimulated by conditioned medium of adipose-derived stem cells is enhanced by hypoxia: evidence of increased growth factor secretion, Biomed. Res., 2010, 31 (1), 26-34, or in Bunnell et al., Adipose-derived stem cells: Isolation, expansion and differentiation, Methods, 2008, 45, 115-120. ADSCs may be isolated from human subcutaneous adipose tissue acquired from healthy subjects who underwent effective liposuction.

In the context of the invention, MSCs, more preferably ADSCs, may be autologous or allogenic.

In an embodiment, MSCs, more preferably ADSCs, are autologous, i.e., derived from the same individual.

In an embodiment, MSCs, more preferably ADSCs, are allogenic, i.e., derived from another individual belonging to the same species, although being genetically different.

In one preferred embodiment of the invention, following the isolation, the isolated MSCs, more preferably ADSCs, are expanded. The expansion is usually performed by culturing the isolated cells in control medium until they become confluent, i.e., a cell confluence of about ≥70%.

This is considered the first passage. In the scope of the invention, the expansion is performed not more than 10 passages, preferably not more than 8 passages, more preferably not more than 5 passages, and yet more preferably not more than 3 passages.

In an embodiment, isolated and expanded MSCs, more preferably ADSCs, may be cryopreserved in a cryopreservation medium.

Cryopreservation media are known in the art, for example, as disclosed in Yong et al., Biopreserv. Biobanking, 13 (4), 231-239. Examples of cryopreservation media are a combination of 80% (v/v) of foetal bovine serum, 10% (v/v) of dimethyl sulfoxide (DMSO), and 10% (v/v) of Dulbecco's Modified Eagle's Medium/Nutrient Mixture F-12 (DMEM/Ham's F-12), and a combination of 90% (v/v) of foetal bovine serum and 10% (v/v) of DMSO.

The concentration of isolated and expanded MSCs, more preferably ADSCs, in the cryopreservation medium is usually comprised between 10⁵ and 10⁸, preferably between 10⁶ and 5×10⁷, more preferably between 5×10⁶ and 10⁷ cells/mL of cryopreservation medium.

The therapeutically effective dose of MSCs, more preferably ADSCs, or of isolated and expanded MSCs, more preferably ADSCs, in the composition of the invention is usually comprised between about 4×10⁴ and about 15×10⁷, preferably, between about 10⁵ and about 10×10⁷, more preferably between about 2.5×10⁵ and about 5×10⁷, more preferably between about 5×10⁵ and about 2.5×10⁷, more preferably between about 7.5×10⁵ and about 10⁷, more preferably between about 1×10⁶ and about 20×10⁶, more preferably between about 10⁶ and about 7.5×10⁶ s, more preferably between about 1.5×10⁶ and about 5×10⁶, more preferably about 2×10⁶, and yet more preferably about 10⁶ cells, preferably ADSCs, per mL of the pharmaceutically acceptable vehicle.

### ATP

The composition of the invention comprises an, preferably therapeutically effective, amount of adenosine triphosphate (ATP).

ATP is classified as a nucleoside triphosphate, which indicates that it consists of three components: a nitrogenous base (adenine), the sugar ribose, and the triphosphate, wherein the adenine is attached by the 9-nitrogen atom to the 1′-carbon atom of the sugar ribose, which in turn is attached at the 5′-carbon atom of the sugar to a triphosphate group.

ATP is an organic compound that provides energy to drive and support many processes in living cells, such as muscle contraction, nerve impulse propagation, condensate dissolution, and chemical synthesis.

In an embodiment, ATP is not encapsulated.

In an embodiment, ATP is encapsulated.

In the scope of the invention, encapsulated means that ATP is microencapsulated or nanoencapsulated by polymers or gels, in a microemulsion, or as liposomes.

Polymers suitable for the encapsulation of ATP are, for example, poly(α-hydroxyacids), such as polylactic acid (PLA) and polyglycolic acid (PGA), and copolymers thereof (PLGA), PEG, copolymers of PEG and PLGA, and poly(ε-caprolactone) (PCE).

Gels suitable for the encapsulation of ATP are, for example, hyaluronic acid and salts thereof, cellulose derivatives, gellan gum, alginates and polyvinyl alcohol.

Microemulsion may contain oily components such as ethyl oleate, soybean oil, castor oil, soybean phosphatidylcholine, surfactants and co-surfactants such as glycol derivatives, and aqueous components.

The preparation of encapsulated ATP may be carried out according to well-known processes disclosed in the prior art, such as emulsification-solvent removal processes, coacervation, spray drying, or by using of supercritical fluids, as disclosed in Microencapsulation, Methods and Industrial Applications, Second Edition, S. Benita Ed., 2006, Taylor & Francis, Boca Raton (FL), pages 1-53.

In a preferred embodiment ATP is liposomated ATP, i.e., encapsulated as liposomes, which means that ATP is liposomated, in other words, liposomes comprise ATP.

In embodiment, ATP is a mixture of encapsulated ATP and non-encapsulated ATP.

In a preferred embodiment, ATP is a mixture of liposomated ATP and non-liposomated ATP. In an embodiment, ATP comprises between 10 wt % and 50 wt %, preferably between 20 wt % and 40 wt %, more preferably between 25 wt % and 35 wt %, and yet more preferably about 30 wt % of liposomated ATP, and between 50 wt % and 90 wt %, preferably between 60 wt % and 80 wt %, more preferably between 65 wt % and 75 wt %, and yet more preferably about 70 wt % of non-liposomated ATP.

Liposomated ATP may be prepared according to prior art procedures, such as disclosed, for example, in international patent application WO-A-03/096973.

In an embodiment, ATP is in the form of salt, and hydrates thereof, wherein the cations are usually sodium, calcium, or magnesium.

In the present invention, the salt of ATP is generally selected from monosodium salt, disodium salt, calcium salt, magnesium salt, and hydrates thereof, preferably from monosodium salt, disodium salt, and hydrates thereof, and more preferably the salt of ATP is the disodium salt hydrate.

ATP is commercially available through companies such as, for example, Sigma-Aldrich.

The therapeutically effective dose of ATP in the composition of the invention is comprised, expressed in concentration of ATP, between about 10 μM and about 20 mM, preferably between about 20 μM and about 10 mM, preferably between about 30 μM and about 1 mM, more preferably between about 40 μM and about 750 μM, more preferably between about 50 μM and about 500 μM, more preferably between about 50 μM and about 200 μM or about 250 μM, more preferably between about 75 μM and about 200 μM or about 250 μM, and yet more preferably about 100 μM or 100 μM.

If a salt of ATP is used to prepare the composition of the invention, the skilled person in the art may perform the calculation to determine the therapeutic effective dose of ATP from the specific salt.

### Pharmaceutically Acceptable Vehicle

The composition of the invention comprises a pharmaceutically acceptable vehicle.

The pharmaceutically acceptable vehicle is usually selected from hypothermic biopreservation media and infusion solutions.

A hypothermic biopreservation media is a pharmaceutically acceptable vehicle useful in preserving cells in a viable condition at a temperature comprised between 2 °C and 8 °C, prior to administration to a patient, as disclosed, for example, in Petrenko et al., Stem Cells Int., 2019, article ID 5909524.

Examples of biopreservation media that can be used include HypoThermosol®, Hypo Thermosol® FRS Preservation Media, NutriStor® Cold Storage Solution, VIASPAN®, SP-1, Belzer UW® Cold Storage, Celsior, Chillprotect, or Rokepie®.

An infusion solution is a sterile solution intended for administration into the bodily tissues.

Examples of infusion solutions that can be used include Ringer's solution, Ringer's lactate solution, Hartmann's lactate solution, saline solution, glucose solution, albumin solution, and mixtures thereof.

Ringer's solution is an isotonic intravenous solution, which comprises potassium chloride, sodium chloride, calcium chloride dihydrate, and water for injection. Ringer's lactate solution further comprises sodium lactate.

Hartmann's lactate solution is an isotonic intravenous solution, which comprises sodium chloride, potassium chloride, calcium chloride dihydrate, sodium lactate, and water for injection.

In an embodiment, the pharmaceutically acceptable vehicle is selected from Hypo Thermosol®, HypoThermosol® FRS Preservation Media, Ringer's solution, Ringer's lactate solution, Hartmann's lactate solution, saline solution, glucose solution, albumin solution, and mixtures thereof.

In a preferred embodiment the pharmaceutically acceptable vehicle is selected from Hypo Thermosol®, HypoThermosol® FRS Preservation Media, a combination of Ringer's lactate solution and albumin, and a combination of saline solution and glucose.

In a more preferred embodiment, the pharmaceutically acceptable vehicle is selected from HypoThermosol®, and Hypo Thermosol® FRS Preservation Media, and in a yet more preferred embodiment, it is HypoThermosol® FRS Preservation Media.

Hypo Thermosol® and Hypo Thermosol® FRS Preservation Media are commercially available products through, for example, the company Sigma-Aldrich.

Hypo Thermosol® is a well-known and well-established hypothermic biopreservation media in the art, whose development and composition as hypothermic blood substitute solution is already disclosed in Taylor et al., Circulation, 1995, 91 (2), 431-44. Its composition is further disclosed, for example, in Ostrowska et al., Arch. Toxicol., 2009, 83 (5), 493-502. According to these disclosures Hypo Thermosol® comprises the components shown in Table I:

TABLE I

| Component | Value |
|---|---|
| Na+ | 100.0 mM |
| K+ | 42.5 mM |
| Ca2+ | 0.05 mM |
| Mg2+ | 5.0 mM |
| Cl− | 17.1 mM |
| H2PO4− | 10 mM |
| HCO3− | 5.0 mM |
| HEPES | 25.0 mM |
| Lactobionate | 100.0 mM |
| Sucrose | 20.0 mM |
| Mannitol | 20.0 mM |
| Glucose | 5.0 mM |
| Dextran-40 | 6% |
| Adenosine | 2.0 mM |
| Glutathione | 3.0 mM |

Therefore, the qualitative composition of Hypo Thermosol® comprises Na⁺, K⁺, Ca²⁺, Mg²⁺, Cl⁻, H₂PO₄⁻, HCO₃⁻, HEPES (i.e., 4-(2-hydroxyethyl)-1-piperazineethanesulfonic acid), lactobionate, sucrose, mannitol, glucose, dextran-40 (MW=40,000 Da), adenosine and glutathione.

The composition of Hypo Thermosol® FRS Preservation Media is disclosed, for example, in Ostrowska et al., op. cit., and in WO-A-2006/125991. Hypo Thermosol® FRS Preservation Media further comprises Trolox (i.e., 6-hydroxy-2,5,7,8-tetramethylchromane-2-carboxylic acid) in the composition of HypoThermosol®. Therefore, the qualitative composition of HypoThermosol® FRS Preservation Media comprises Na⁺, K⁺, Ca²⁺, Mg²⁺, Cl⁻, H₂PO₄⁻, HCO₃⁻, HEPES, lactobionate, sucrose, mannitol, glucose, dextran-40, adenosine, glutathione, and 6-hydroxy-2,5,7,8-tetramethylchromane-2-carboxylic acid.

### Process for Preparing the Composition for Promoting Hair Growth

The process for preparing the composition for promoting hair growth comprises mixing isolated and expanded MSCs, preferably ADSCs, with ATP, or a salt thereof, and a pharmaceutically acceptable vehicle.

The manufacturing process preferably consists of thawing cryopreserved mesenchymal stem cells, preferably ADSCs, and cell maintenance in the following 24-48 hours and final conditioning in a pharmaceutically acceptable vehicle in which it has been previously added ATP.

The immunophenotype of the mesenchymal stem cells, preferably ADSCs, in the composition of the invention remains stable at least 24 h at a temperature of 4 °C.

### Kit of Parts

A further aspect of the invention refers to a kit of parts comprising:
a. a first vial containing a population of MSCs as defined in the previous aspects of the invention, including any of its preferred embodiments; and
b. a second vial comprising ATP as defined in the previous aspects of the invention, including any of its preferred embodiments;
   wherein the first vial and/or the second vial further comprised a pharmaceutically acceptable vehicle as defined in the previous aspect of the invention, including any of its preferred embodiments. It is further noted that the pharmaceutically acceptable vehicle can be optionally provided in a further vial.

Preferably, the kit of parts further comprises instructions for use specifying the co-administration or sequential application of the MSCs and the ATP for promoting hair growth.

### Use of the Composition for Promoting Hair Growth

Another aspect of the present invention is the composition, the hair growth composition of the invention, or the kit of parts of the invention, for use in promoting hair growth, preferably for use in the prevention and/or treatment of alopecia, preferably for use in the prevention and/or treatment of androgenic alopecia, in a subject in need thereof, preferably in a human subject.

In an embodiment, the composition or the kit of parts is for use in the prevention of androgenic alopecia.

In an embodiment, the composition or the kit of parts is for use in the treatment of androgenic alopecia.

Another aspect of the invention is the cosmetic or non-therapeutic use of said composition or kit of parts for promoting hair growth in a subject in need thereof.

Another aspect of the invention are isolated and expanded mesenchymal stem cells, preferably ADSCs, for use in promoting hair growth, preferably for use in the prevention and/or treatment of androgenic alopecia.

In an embodiment, isolated and expanded MSCs, preferably ADSCs, are for use in the prevention of androgenic alopecia.

In an embodiment, isolated and expanded MSCs, preferably ADSCs, are for use in the treatment of androgenic alopecia.

In an embodiment, the subject suffering from androgenic alopecia is a male subject.

In an embodiment, the subject suffering from androgenic alopecia is a female subject.

The dose of the MSCs, more preferably ADSCs, more preferably isolated and expanded MSCs, preferably isolated and expanded ADSCs, alone or in combination with

ATP may be appropriately decided for each case taking into consideration the patient's degree of androgenic alopecia.

In an embodiment, the dose of MSCs, more preferably ADSCs, more preferably isolated and expanded MSCs, preferably isolated and expanded ADSCs, to be administered is usually comprised between about 10⁵ cells, preferably ADSCs, and about 10⁶ cells, preferably ADSCs, combined with a concentration of ATP comprised between about 10 μM and about 20 mM, in a pharmaceutically acceptable vehicle, per cm² of alopecic area. However, any of the further doses specified herein are also included.

The dose of the composition of the MSCs, more preferably ADSCs, more preferably isolated and expanded MSCs, preferably isolated and expanded ADSCs, alone or in combination with ATP, is usually administered by local application to the skin by intradermal and/or subcutaneous route using an injection.

The dose may be administered once or it may be divided into 2 or more administrations.

The dose of MSCs, more preferably ADSCs, more preferably isolated and expanded MSCs, preferably isolated and expanded ADSCs, alone or in combination with ATP, may be administered at least once more, or twice, or more times, if necessary. In addition, a different number of punctures may be used to apply the composition.

In an embodiment, the active components of the composition, the MSCs, more preferably ADSCs, more preferably isolated and expanded MSCs, preferably isolated and expanded ADSCs and ATP, may be administered separately by local application to the skin by intradermal and/or subcutaneous route using an injection. It is noted that much preferably both components are simultaneously administered in the form of the composition for hair growth of the present invention.

A further embodiment of the invention refers to the use of the composition of the invention in promoting hair growth, preferably for use in the prevention and/or treatment of androgenic alopecia, optionally administered in combination with therapeutically effective amounts of minoxidil, finasteride, or dutasteride.

The Hamilton-Norwood scale, also known as Norwood scale or Norwood-Hamilton scale, is a widely accepted and reproducible classification system for the male pattern hair loss (androgenetic alopecia), which is used to classify the stages of male pattern baldness. The stages are described with a number from I to VII with a type A variant for the cases with anterior involvement and may be assigned to the degree of alopecia.

The approximated dose of isolated and expanded MSCs, preferably ADSCs, alone or in combination with ATP, to be administered is usually as shown in Table II according to the different degree of alopecia in men:

TABLE II

| | | Bald surface | |
|---|---|---|---|
| Alopecia degree | Norwood scale | (cm²) | MSCs dose (cells) |
| Mild | II-III | 18-40 | 2 × 10⁶-4.5 × 10⁶ |
| Moderate | IV | 41-120 | 4.5 × 10⁶-13.5 × 10⁶ |
| Severe | V-VII | 121-200 | 13.5 × 10⁶-22.3 × 10⁶ |

The Ludwig scale is a method of classifying female pattern baldness (androgenic alopecia), and ranges from stages I to III.

The approximated dose of isolated and expanded MSCs, preferably SCsADSCs, alone or in combination with ATP, to be administered is usually as shown in Table III according to the different degree of alopecia in women:

TABLE III

| | | Bald surface | |
|---|---|---|---|
| Alopecia degree | Ludwig scale | (cm²) | MSCs dose (cells) |
| Mild | I | <50 | 11 × 10⁶ |
| Moderate | II | 51-100 | 11 x 10⁶-22 × 10⁶ |
| Severe | III | 101-150 | 22 x 10⁶-33.5 × 10⁶ |

Surprisingly, the combination of isolated and expanded MSCs, preferably ADSCs, and ATP provides an improved, even synergistic, composition for promoting hair growth in in vivo experiments performed in mice, as disclosed in the Examples.

Not only the combination of isolated and expanded MSCs, preferably ADSCs, and ATP promotes hair growth, but examples show that isolated and expanded MSCs, preferably ADSCs, alone dispersed in a pharmaceutically acceptable vehicle, such as, for example, HypoThermosol® FRS Preservation Media, are also capable of promoting hair growth in in vivo experiments performed in mice.

Therefore isolated and expanded MSCs, preferably ADSCs, alone or in combination with non-encapsulated ATP or encapsulated ATP are candidates for promoting hair growth, in particular in AGA. In a preferred embodiment, MSCs, preferably ADSCs, are in combination with non-encapsulated ATP and liposomated ATP.

The present invention may be further defined according to the following clauses:
1.—A composition for promoting hair growth comprising a therapeutically effective amount of isolated and expanded MSCs, preferably adipose derived stem cells (ADSC), a therapeutically effective amount of ATP, and a pharmaceutically acceptable vehicle, wherein the therapeutically effective dose of ATP is comprised, expressed in concentration of ATP, between about 10 μM and about 20 mM.
2.—The composition according to clause 1, wherein the therapeutically effective dose of isolated and expanded is comprised between about 4×10⁴ and about 15×10⁷, preferably, between about 10⁵ and about 10×10⁷, more preferably between about 2.5×10⁵ and about 5×10⁷, more preferably between about 5×10⁵ and about 2.5×10⁷, more preferably between about 7.5×10⁵ and about 10⁷, more preferably 10⁶ and about 7.5×10⁶, more preferably between 1.5×10⁶ and about 5×10⁶, more preferably about 2×10⁶, and yet more preferably about 10⁶ mesenchymal stem cells, preferably ADSCs, per mL of the pharmaceutically acceptable vehicle.
3.—The composition according to clause 1 or 2, wherein ATP is encapsulated.
4.—The composition according to clause 3, wherein ATP is liposomated ATP.
5.—The composition according to clause 1 or 2, wherein ATP is a mixture of encapsulated ATP and non-encapsulated ATP.
6.—The composition according to clause 4, wherein ATP is a mixture of liposomated ATP and non-liposomated ATP.
7.—The composition according to clause 6, wherein ATP comprises between 10 wt % and 50 wt %, preferably between 20 wt % and 40 wt %, more preferably between 25 wt % and 35 wt %, and yet more preferably about 30 wt % of liposomated ATP, and between 50 wt % and 90 wt %, preferably between 60 wt % and 80 wt %, more preferably between 65 wt % and 75 wt %, and yet more preferably about 70 wt % of non-liposomated ATP.
8.—The composition according to any one of clauses 1 to 7, wherein ATP is in the form of salt.
9.—The composition according to clause 6, wherein the salt of ATP is selected from monosodium salt, disodium salt, calcium salt, magnesium salt, and hydrates thereof, preferably from monosodium salt, disodium salt, and hydrates thereof, and more preferably the salt of ATP is the disodium salt hydrate.
10.—The composition according to any one of clauses 1 to 9, wherein the therapeutically effective dose of ATP is comprised, expressed in concentration of ATP, between about 20 UM and about 10 mM, preferably between about 30 μM and about 1 mM, more preferably between about 40 μM and about 750 μM, more preferably between about 50 μM and about 500 μM, more preferably between about 75 μM and about 250 μM, and yet more preferably about 100 μM.
11.—The composition according to any one of clauses 1 to 10, wherein the pharmaceutically acceptable vehicle is selected from hypothermic biopreservation media and infusion solutions.
12.—The composition according to clause 11, wherein the pharmaceutically acceptable vehicle is selected from Hypo Thermosol®, HypoThermosol® FRS Preservation Media, Ringer's solution, Ringer's lactate solution, Hartmann's lactate solution, saline solution, glucose solution, albumin solution, and mixtures thereof.
13.—The composition according to clause 12, wherein the pharmaceutically acceptable vehicle is selected from Hypo Thermosol®, HypoThermosol® FRS Preservation Media, a combination of Ringer's lactate solution and albumin, and a combination of saline solution and glucose.
14.—The composition according to clause 13, wherein the pharmaceutically acceptable vehicle is selected from Hypo Thermosol®, and Hypo Thermosol® FRS Preservation Media.
15.—The composition according to clause 14, wherein the pharmaceutically acceptable vehicle is HypoThermosol® FRS Preservation Media.
16.—The composition according to any one of clauses 1 to 15, wherein it comprises:
   a) a therapeutically effective dose of isolated and expanded mesenchymal stem cells, preferably ADSCs, comprises between about 4×10⁴ and about 15×10⁷, preferably, between about 10⁵ and about 10×10⁷, more preferably between about 2.5×10⁵ and about 5×10⁷, more preferably between about 5×10⁵ and about 2.5×10⁷, more preferably between about 7.5×10⁵ and about 10⁷, more preferably 10⁶ and about 7.5×10⁶, more preferably between 1.5×10⁶ and about 5×10⁶, more preferably about 2×10⁶, and yet more preferably about 10⁶ mesenchymal stem cells, preferably ADSCs, per mL of the pharmaceutically acceptable vehicle, and
   b) a therapeutically effective dose of ATP comprised, expressed in concentration of ATP, between about 10 μM and about 20 mM, preferably between about 20 μM and about 10 mM, preferably between about 30 μM and about 1 mM, more preferably between about 40 μM and about 750 μM, more preferably between about 50 μM and about 500 μM, more preferably between about 75 μM and about 250 μM, and yet more preferably about 100 μM, combined in a pharmaceutically acceptable vehicle.
17.—The composition according to clause 16, wherein it comprises 10⁶ mesenchymal stem cells, preferably ADSCs, per mL of HypoThermosol® FRS Preservation Media as pharmaceutically acceptable vehicle, and a concentration of ATP comprised between about 75 μM and about 250 μM, preferably about 100 μM.
18.—The composition according to any one of clauses 1 to 17 for use in promoting hair growth or the cosmetic use of the composition according to any one of embodiments 1 to 17 for promoting hair growth.
19.—The composition according to clause 18 for use in the prevention of androgenic alopecia and/or treatment of a subject suffering from androgenic alopecia
20.—The composition according to clause 19 for use in the prevention of androgenic alopecia.
21.—The composition according to clause 19 for use in the treatment of a subject suffering from androgenic alopecia.
22.—The composition according to any one of clauses 19 to 21, wherein the subject suffering from androgenic alopecia is a male subject.
23.—The composition according to any one of clauses 19 to 21, wherein the subject suffering from androgenic alopecia is a female subject.
24.—The composition according to clause 19 for use in the prevention of androgenic alopecia and/or treatment of a subject suffering from androgenic alopecia administered in combination with therapeutically effective amounts of minoxidil, finasteride, or dutasteride.
25.—Isolated and expanded mesenchymal stem cells, preferably ADSCs, for use in promoting hair growth.
26.—Isolated and expanded mesenchymal stem cells, preferably ADSCs, for use in the prevention of androgenic alopecia and/or treatment of androgenic alopecia.
27.—Isolated and expanded mesenchymal stem cells, preferably ADSCs, for use according to embodiment 26 in the prevention of androgenic alopecia.
28.—Isolated and expanded mesenchymal stem cells, preferably ADSCs, for use according to clause 26 in the treatment of a subject suffering from androgenic alopecia.
29.—Isolated and expanded mesenchymal stem cells, preferably ADSCs, for use according to any one of clauses 26 to 28 wherein the subject suffering from androgenic alopecia is a male subject.
30.—Isolated and expanded mesenchymal stem cells, preferably ADSCs, for use according to any one of embodiments 26 to 28, wherein the subject suffering from androgenic alopecia is a female subject.
31.—The composition for use or the cosmetic use according to embodiment 18, wherein the dose of isolated and expanded mesenchymal stem cells, preferably ADSCs, to be administered is comprised between about 10⁵ mesenchymal stem cells, preferably ADSCs, and about 10⁶ mesenchymal stem cells, preferably ADSCs, combined with a concentration of ATP comprised between about 10 μM and about 20 mM, in a pharmaceutically acceptable vehicle per cm² of alopecic area.
32.—Isolated and expanded mesenchymal stem cells, preferably ADSCs, for use according to clause 26 wherein the isolated and expanded mesenchymal stem cells, preferably ADSCs, are administered in combination with therapeutically effective amounts of minoxidil, finasteride, or dutasteride.

The present invention may be further defined according to the following clauses:
1.—A composition for promoting hair growth comprising a therapeutically effective amount of isolated and expanded adipose derived stem cells (ADSC), a therapeutically effective amount of ATP, and a pharmaceutically acceptable vehicle.
2.—The composition according to clause 1, wherein the therapeutically effective dose of isolated and expanded ADSCs is comprised between about 4×10⁴ ADSCs and about 15×10⁷ ADSCs per mL of the pharmaceutically acceptable vehicle.
3.—The composition according to clause 1 or 2, wherein the therapeutically effective dose of ATP is comprised, expressed in concentration of ATP, between about 10 μM and about 20 mM.
4.—The composition according to any one of clauses 1 to 3, wherein the pharmaceutically acceptable vehicle is selected from hypothermic biopreservation media and infusion solutions.
5.—The composition according to clause 4, wherein the pharmaceutically acceptable vehicle is selected from Hypo Thermosol®, Hypo Thermosol® FRS Preservation Media, Ringer's solution, Ringer's lactate solution, Hartmann's lactate solution, saline solution, glucose solution, albumin solution, and mixtures thereof.
6.—The composition according to clause 5, wherein the pharmaceutically acceptable vehicle is selected from Hypo Thermosol®, HypoThermosol® FRS Preservation Media, a combination of Ringer's lactate solution and albumin, and a combination of saline solution and glucose.
7.—The composition according to clause 6, wherein the pharmaceutically acceptable vehicle is selected from Hypo Thermosol®, and Hypo Thermosol® FRS Preservation Media.
8.—The composition according to clause 7, wherein the pharmaceutically acceptable vehicle is Hypo Thermosol® FRS Preservation Media.
9.—The composition according to any one of clauses 1 to 9, wherein it comprises:
   a) a therapeutically effective dose of isolated and expanded ADSCs comprises between about 4×10⁴ ADSCs and about 15×10⁷ ADSCs per mL of the pharmaceutically acceptable vehicle, and
   b) a therapeutically effective dose of ATP comprised, expressed in concentration of ATP, between about 10 μM and about 20 mM, combined in a pharmaceutically acceptable vehicle.
10.—The composition according to clause 9, wherein it comprises 10⁶ ADSCs per mL of Hypo Thermosol® FRS Preservation Media as pharmaceutically acceptable vehicle, and a concentration of ATP comprised between about 75 μM and about 250 μM.
11.—The composition according to any one of clauses 1 to 10 for use in promoting hair growth.
12.—The composition according to any one of clauses 1 to 10 for use in the prevention of androgenic alopecia and/or treatment of a subject suffering from androgenic alopecia 13.—Isolated and expanded ADSCs for use in promoting hair growth.
14.—Isolated and expanded ADSCs for use in the prevention of androgenic alopecia and/or treatment of androgenic alopecia.
15.—The composition for use according to clause 11 or the isolated and expanded ADSCs according for use according to clause 13, wherein the dose of isolated and expanded ADSCs to be administered is comprised between about 10⁵ ADSCs and about 10⁶ ADSCs combined with a concentration of ATP comprised between about 10 μM and about 20 mM, in a pharmaceutically acceptable vehicle per cm² of alopecic area.

### EXAMPLES

### Preparative Example: Preparation of Isolated and Expanded Adipose Derived Stem Cells

Isolated and expanded adipose derived stem cells of the invention were prepared from adipose tissue collected by liposuction aspiration, following substantially the process disclosed in Bunnell et al., Adipose-derived stem cells: Isolation, expansion and differentiation, Methods, 2008, 45, 115-120.

ADSCs were cryopreserved in a cryopreservation medium comprising a combination of 90% (v/v) of foetal bovine serum and 10% (v/v) of DMSO.

### Examples 1-4: Compositions for Promoting Hair Growth

The process for preparing the composition for promoting hair growth comprises thawing cryopreserved isolated and expanded ADSCs, cell maintenance in the following 24-48 hours, and mixing with ATP, or a salt thereof, in a pharmaceutically acceptable vehicle.

Examples of prepared compositions of the invention are shown in Table IV:

TABLE IV

| Component | Ex. 1 | Ex. 2 | Ex. 3 | Ex. 4 |
|---|---|---|---|---|
| Isolated and | 2 × 10⁶ | 10 × 10⁶ | 10 × 10⁶ | 20 × 10⁶ |
| expanded ADSCs | | | | |
| ATP | — | — | 100 μM | 100 μM |
| ATP (30% | 100 μM | 100 μM | — | — |
| liposomated ATP) | | | | |
| Pharmaceutically | q.s. 2 mL | q.s. 10 mL | q.s. 10 mL | q.s. 20 mL |
| acceptable | | | | |
| vehicle | | | | |

The pharmaceutically acceptable vehicle used was HypoThermosol® FRS Preservation Media (HTS) (Sigma-Aldrich).

The compositions of Examples 1 and 2 were suitable for male pattern hair loss, and the compositions of Examples 3 and 4 for female pattern hair loss.

### Example 5: Use of Compositions for Promoting Hair Growth

The compositions for promoting hair growth of the invention were tested in vivo in mice, according to the following procedure.

### 1.—Experimental Animals

A total of 45 6-week-old C57BL6 mice (males) from Charles River Laboratories were used.

### 2.—AGA Model

The animals were acclimatized for 6 days, and they were administered 1 mg of DHT (5α-androstan-17β-ol-3-one, Sigma) in 100 μL of 50% DMSO (Merck)/50% Isotonic Saline Serum (Fresenius Kabi) subcutaneously for 28 days (5 days a week), resulting 19 administrations, instead of 20, because the dose was omitted on day 21, when the final assessment of hair growth took place. The administration of DHT began at day-4 of the testing.

A delay in the hair growth is observed, hence the model was suitable for assessing the effect of the different treatments.

### 3.—Groups

The groups of mice used in the testing are shown in Table V:

TABLE V

| | | Number |
|---|---|---|
| Group | Definition | of mice |
| 1 | Administration of HTS (Control) | n = 5 |
| 2 | Administration of ADSCs-high dose 4 × 10⁶ and | n = 10 |
| | HTS | |
| 3 | Administration of ATP (30% liposomated ATP) and | n = 5 |
| | HTS | |
| 4 | Administration of non-encapsulated ATP and HTS | n = 5 |
| 5 | Administration of ADSCs-low dose 1 × 10⁶ + ATP | n = 10 |
| | (30% liposomated ATP) and HTS | |
| 6 | Administration of ADSCs-low dose 1 × 10⁶ + non- | n = 10 |
| | encapsulated ATP and HTS | |

A group of n=5 mice was reserved.

The concentration of ATP in Groups 3, 4, 5 and 6 was 100 μM.

### 4.—Depilation

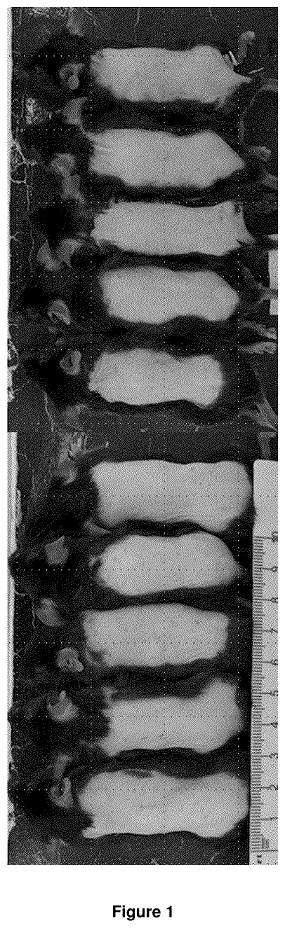

After 4 days of administration of the DHT solution (day 0), the target area was depilated using an electric shaver in the area delimited by a template. Subsequently, depilatory cream was applied with the aim of uniformizing the beginning of the anagen phase in all mice. This procedure was performed using inhalation sedation with 2% Isoflurane, maintaining the body temperature of the animals with an electric blanket and an infrared lamp. (FIG. 1).

### 5.—Administration of the Compositions

The compositions were administered intradermally on day 0 and on day 3 of the testing in 18 injections of 10 μL each with a 30 G needle distributed over a defined shaving area of 4.5×2 cm (9 cm² divided into an upper, central and lower quadrant of 2×1.5 cm each). In each quadrant, 6 administrations were carried out.

The dosage to the ADSC-high dose group consisted of two injections of 2×10⁶ cells and vehicle.

The dosage to the ADSCs-low dose groups (with liposomated ATP and non-encapsulated ATP) consisted of two injections of 0.5×10⁶ cells and vehicle.

### 6.—Assessment of Hair Growth

The macroscopic assessment of hair growth was carried out by blind assessment through photographs of each group at different times, assigning percentages based on hair growth classified into different levels of repopulation (nil, poor, moderate, intense and full) on day 21 in the case of males.

### 7.—Results

In Table VI are shown the results obtained for male mice at day 21, expressed in % of mice:

TABLE VI

| Group | Ni | Poor | Moderate | Intense | Full |
|---|---|---|---|---|---|
| 1 | 20 | 20 | 20 | 40 | — |
| HTS | | | | | |
| 2 | — | — | 30 | 50 | 20 |
| ADSCs high | | | | | |
| 3 | — | 20 | 20 | 40 | 20 |
| ATP liposomated | | | | | |
| 4 | — | — | 20 | 80 | — |
| ATP non-encapsulated | | | | | |
| 5 | — | — | — | 50 | 50 |
| ADSCs low + | | | | | |
| ATP liposomated | | | | | |
| 6 | — | — | | 70 | 30 |
| ADSCs low + | | | | | |
| ATP non-encapsulated | | | | | |

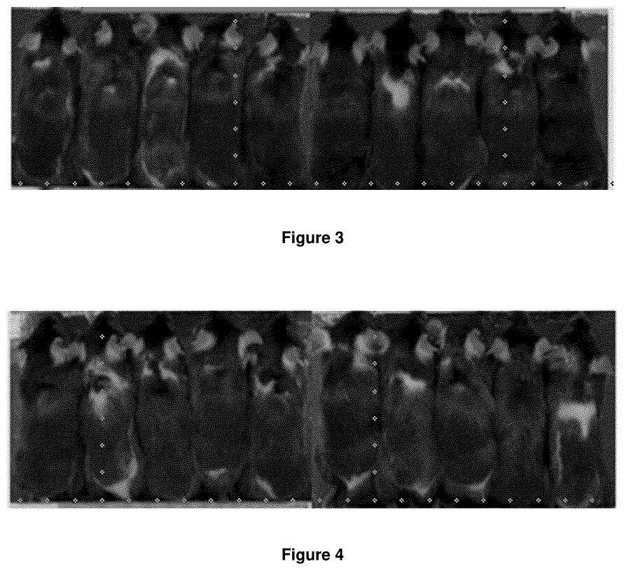

It can be observed that the low dose of isolated and expanded ADSCs combined with liposomated ATP (Group 5, FIG. 3), and low dose of isolated and expanded ADSCs combined with non-encapsulated ATP (Group 6, FIG. 4) provided 100% of intense/full hair growth, and that isolated and expanded ADSCs alone provided 70% of intense/full hair growth in male mice at day 21.

The effects obtained with the combination of isolated and expanded ADSCs, and ATP are better than the sum of the separated components, considering that the tested concentration of ADSCs alone is 4 times higher than the concentration of the low dose of ADSCs (4×10⁶ vs 1× 10⁶).

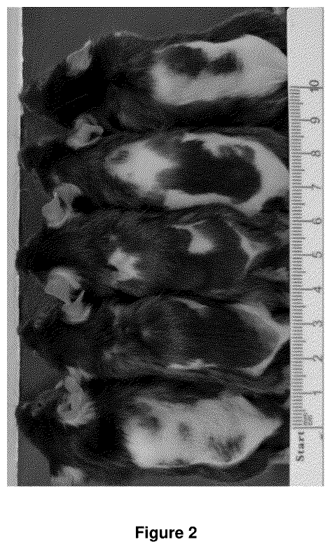

It can be observed also that all treatments with isolated and expanded ADSCs showed better hair growth effect than the treatment with the pharmaceutically acceptable vehicle alone HTS (Group 1, FIG. 2).

1. A composition for promoting hair growth comprising between about 5×10⁵ and about 2.5×10⁷ isolated and expanded adipose-derived stem cells (ADSCs) or bone marrow-derived mesenchymal stem cells (MSCs), 50 μM to 250 μM adenosine triphosphate (ATP), and a pharmaceutically acceptable vehicle.

2. The composition according to claim 1, wherein the composition comprises between about 1×10⁶ and about 7.5×10⁶ isolated and expanded ADSCs or bone marrow-derived MSCs.

3. The composition according to claim 1, wherein the composition comprises about 1×10⁶ isolated and expanded ADSCs or bone marrow-derived MSCs.

4. The composition according to claim 1, wherein the composition comprises about 75 μM to 200 μM ATP.

5. The composition according to claim 1, wherein the composition comprises about 100 PM ATP.

6. The composition according to claim 1, wherein the isolated and expanded ADSCs or bone marrow-derived MSCs are present in the composition in a concentration between about 1×10⁶ cells per mL and about 5×10⁶ cells per mL of the pharmaceutically acceptable vehicle.

7. The composition according to claim 1, wherein the isolated and expanded ADSCs or bone marrow-derived MSCs are present in the composition in a concentration of about 1×10⁶ cells per mL of the pharmaceutically acceptable vehicle.

8. The composition according to claim 1, wherein the composition comprises about 1×10⁶ isolated and expanded ADSCs and about 100 μM ATP.

9. The composition according to claim 1, wherein the ATP is liposomated ATP.

10. The composition according to claim 1, wherein the pharmaceutically acceptable vehicle is selected from hypothermic biopreservation media and infusion solutions.

11. A method for prevention or treatment of alopecia comprising administering the composition according to claim 1 to a subject in need thereof.

12. The method according to claim 11, wherein the alopecia is androgenic alopecia.

13. The method according to claim 12, wherein the subject is a male subject.

14. The method according to claim 12, wherein the subject is a female subject.

15. A method for promoting hair growth comprising applying the composition according to claim 1 to a subject in need thereof.
